(19) United States
(12) Patent Application Publication
(10) Pub. No.: US 2026/0008036 A1
(43) Pub. Date: Jan. 8, 2026

(54) PREPARATION METHOD OF ION CATALYST MATERIAL FOR PET CHEMICAL RECYCLING AND PET CHEMICAL RECYCLING METHOD

(71) Applicant: NAN YA PLASTICS CORPORATION, Taipei City (TW)

(72) Inventors: Ching-Yao Yuan, Taipei City (TW); Sen-Huang Hsu, Taipei City (TW)

(73) Assignee: NAN YA PLASTICS CORPORATION, Taipei City (TW)

(21) Appl. No.: 19/323,993

(22) Filed: Sep. 9, 2025

Related U.S. Application Data
(63) Continuation-in-part of application No. 17/826,195, filed on May 27, 2022.

(30) Foreign Application Priority Data
Oct. 7, 2021 (TW) 110137404

(51) Int. Cl. B01J 31/02; B01J 21/18; B01J 37/02; B01J 37/04; C07C 67/297; C08J 11/28

(52) U.S. Cl. CPC B01J 31/0281; B01J 21/18; B01J 31/0298; B01J 37/0219; B01J 37/04; C07C 67/297; C08J 11/28; B01J 2231/005; B01J 2531/002; B01J 2531/26; C08J 2367/02

(57) ABSTRACT

Provided are a preparation method of an ion catalyst material for PET chemical recycling and a PET chemical recycling method. The preparation method of an ion catalyst material for PET chemical recycling includes the following. A metal chloride is added to an alkylimidazole-chloride ionic liquid to form a bisalkylimidazole-metal tetrachloride ionic liquid that is grafted on a porous carrier.

### CROSS-REFERENCE TO RELATED APPLICATION

This application is a continuation-in-part application of and claims the priority benefit of U.S. application Ser. No. 17/826,195, filed on May 27, 2022, which claims the priority benefit of Taiwan application serial no. 110137404, filed on Oct. 7, 2021. The entirety of each of the above-mentioned patent applications is hereby incorporated by reference herein and made a part of this specification.

### BACKGROUND

### Technical Field

The disclosure relates to a preparation method of an ion catalyst material for PET chemical recycling and a PET chemical recycling method, and in particular, to a preparation method of an ion catalyst material for PET chemical recycling and a PET chemical recycling method which achieve decolorization and depolymerization and facilitate recycling.

### Related Art

Polyethylene terephthalate (PET) is one of the most widely used plastics and may be used in rigid packaging materials for beverages and food, as well as textile fabrics. In view of the global trend of environmental protection and pollution prevention, many governments require both public agencies and private businesses to use recycled PET products, and major international brands have set targets for using recycled materials. These measures have promoted the development of PET recycling technology.

With the increasing complexity of commercially available PET packaging materials and the widespread use of PET, recycling operations have become more and more complex. Thus, chemical recycling for recycling and depolymerization of PET waste plastics have received attention. Recently, the closed-loop chemical recycling technology for PET has developed rapidly around the globe. Techniques such as solvent-assisted chemical degradation, biodegradation, and microwave-assisted chemical degradation have reached the trial mass production stage, in which different types of PET waste plastics, such as packaging materials and textile fabrics, can be processed. However, in most of the current PET chemical recycling technologies, depolymerization and decolorization need to be separately performed. In addition, recycling of a catalyst is not easy, thereby significantly increasing the cost.

Based on the above, it has become a hot research topic to develop a chemical recycling technique for the recycling and depolymerization of PET waste plastics to make it possible to perform depolymerization and decolorization at the same time and to recycle a catalyst.

### SUMMARY

The disclosure provides a preparation method of an ion catalyst material for PET chemical recycling and a PET chemical recycling method, in which decolorization and depolymerization can be achieved and recycling can be facilitated.

A preparation method of an ion catalyst material for PET chemical recycling according to the disclosure includes the following. A metal chloride is added to an alkylimidazole-chloride ionic liquid to form a bisalkylimidazole-metal tetrachloride ionic liquid that is grafted on a porous carrier.

In an embodiment of the disclosure, the metal chloride includes iron chloride, zinc chloride, or cobalt chloride.

In an embodiment of the disclosure, the porous carrier includes nano iron oxide, silicon oxide, or activated carbon.

In an embodiment of the disclosure, a ratio of the metal chloride to the alkylimidazole-chloride ionic liquid is 1 to 2.

In an embodiment of the disclosure, the porous carrier has a graft rate of 6 wt % to 10 wt %.

A PET chemical recycling method according to the disclosure includes the following. A PET chemical recycling ion catalyst material prepared by the above-mentioned preparation method of an ion catalyst material for PET chemical recycling is added to PET waste and ethylene glycol, followed by heating and stirring to obtain depolymerized and decolorized bis(2-hydroxyethyl)terephthalate (BHET). Then, the ion catalyst material for PET chemical recycling is filtered and recycled.

In an embodiment of the disclosure, the PET waste includes a packaging material or a textile fabric.

In an embodiment of the disclosure, a ratio of the PET waste to the ethylene glycol is 1:3 to 1:6.

In an embodiment of the disclosure, an addition amount of the ion catalyst material for PET chemical recycling is 0.5% to 2%.

In an embodiment of the disclosure, a heating temperature is 180° C. to 210° C., a stirring speed is 100 rpm to 150 rpm, and stirring time is 3 hours to 6 hours.

Based on the above, in the preparation method of an ion catalyst material for PET chemical recycling according to the disclosure, a porous material is used as a carrier so as to improve the decolorization ability with respect to PET waste (especially a textile fabric), and a bisalkylimidazole-metal tetrachloride ionic liquid is used so as to improve the depolymerization ability. In this way, in the disclosure, decolorization and depolymerization can be achieved and recycling can be facilitated by a simple manufacturing process.

### DESCRIPTION OF EMBODIMENTS

Hereinafter, embodiments of the disclosure will be described in detail. However, these embodiments are illustrative, and the disclosure is not limited thereto.

In the present specification, a range represented by “a numerical value to another numerical value” is a schematic representation for avoiding listing all of the numerical values in the range in the specification. Therefore, the recitation of a specific numerical range covers any numerical value in the numerical range and a smaller numerical range defined by any numerical value in the numerical range, as is the case with any numerical value and a smaller numerical range thereof in the specification.

The disclosure provides a preparation method of an ion catalyst material for PET chemical recycling. The preparation method includes the following. A metal chloride is added to an alkylimidazole-chloride ionic liquid to form a bisalkylimidazole-metal tetrachloride ionic liquid that is grafted on a porous carrier.

In the present embodiment, the metal chloride may include iron chloride, zinc chloride, or cobalt chloride. However, the disclosure is not limited thereto. A ratio of the metal chloride to the alkylimidazole-chloride ionic liquid is 1 to 2. The porous carrier may include nano iron oxide, silicon oxide, or activated carbon. However, the disclosure is not limited thereto. The porous carrier has a graft rate of about 6 wt % to 10 wt %, and the graft rate is the ratio of weight of the bisalkylimidazole-metal tetrachloride ionic liquid to weight of the porous carrier.

According to one embodiment of the present disclosure, zinc chloride is used as the metal chloride, and 1-Butyl-3-methylimidazolium chloride ([BMIM]Cl) is used as the alkylimidazole-chloride ionic liquid. The zinc chloride is added to a 1-Butyl-3-methylimidazolium chloride at a temperature of 60° C. to 120° C. for 1 hour to 6 hours to form a bisalkylimidazole-zinc tetrachloride ionic liquid that is grafted on a porous carrier.

When the reaction temperature is below 60° C., the metal chloride might not react with the alkylimidazole-chloride. When the reaction temperature is more than 120° C., the metal chloride and the alkylimidazole-chloride might volatilize to form irritating and corrosive fumes. Specifically, the vapor of ZnCl₂ is toxic; in a water-containing environment, ZnCl₂ can decompose to produce hydrogen chloride (HCl) gas, which is highly corrosive and can damage the respiratory tract and lungs after inhalation. When the reaction time is below 1 hour, the metal chloride might not completely react with the alkylimidazole-chloride, resulting in longer required time for depolymerization and lower depolymerization efficiency during PET chemical recycling. When the reaction time is more than 6 hours, the cost rises.

According to one embodiment of the present disclosure, a reaction of adding the zinc chloride to the 1-Butyl-3-methylimidazolium chloride to form the bisalkylimidazole-zinc tetrachloride ionic liquid is represented by following Formula 1.

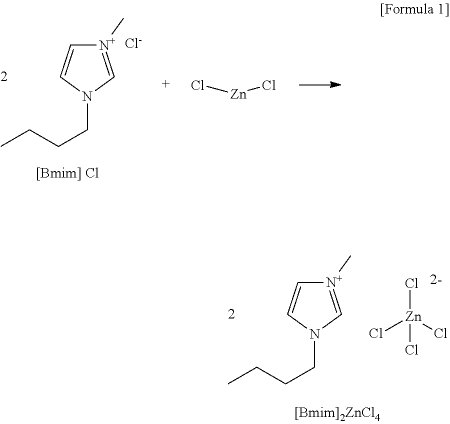

The disclosure also provides a PET chemical recycling method including the following. A PET chemical recycling ion catalyst material prepared by the above-mentioned preparation method of an ion catalyst material for PET chemical recycling is added to PET waste and ethylene glycol, followed by heating and stirring to obtain depolymerized and decolorized bis(2-hydroxyethyl)terephthalate (BHET). Then, the ion catalyst material for PET chemical recycling is filtered and recycled.

In the present embodiment, the PET waste may include a packaging material or a textile fabric. However, the disclosure is not limited thereto. A ratio of the PET waste to the ethylene glycol is, for example, 1:3 to 1:6. An addition amount of the ion catalyst material for PET chemical recycling is, for example, 0.5% to 2%. A heating temperature is, for example, 180° C. to 210° C., a stirring speed is, for example, 100 rpm to 150 rpm, and stirring time is, for example, 3 hours to 6 hours.

By the PET chemical recycling method according to the disclosure, in an experimental example for a textile fabric, after being subjected to depolymerization, BHET purification, and polymerization, the textile fabric was subjected to gel permeation chromatography (GPC) testing, in which depolymerization efficiency was measured as 93%, and chromatic values were measured as an L value of 85 to 90, an a value of −1 to 1, and a b value of 1 to 4.

Detailed embodiments of the present invention will be further specified below in combination with the examples, but implementation and protection of the present invention are not limited thereto. It needs to be indicated that the processes not described very specifically below, if any, shall be achieved or understood to those skilled in the art by referring to the prior art. The used reagents or instrument not marked a manufacturer should be regarded as conventional products available commercially.

The condition for the process for PET chemical recycling of each Example is summarized in the following Table 1.

TABLE 1

| | Catalyst | Depolymerization and Decolorization Test | | | | |
|---|---|---|---|---|---|---|
| | Material | | | | | |
| | Graft | Depolymerization | | Chromatic Values | | |
| | Rate | | Efficiency | L > 80 | a ± 1 | b + 4 |
| No. | (%) | Time | (%) | L | a | b |
| Comparative | 11.4 | 2 | 85 | 80 | 1.16 | 6.46 |
| Example 1 | | | | | | |
| Comparative | 10.8 | 2 | 86 | 83 | −0.96 | 5.43 |
| Example 2 | | | | | | |
| Comparative | 10.1 | 2 | 89 | 83 | −1.01 | 4.92 |
| Example3 | | | | | | |
| Example 1 | 10.0 | 2 | 92 | 83 | −0.65 | 3.63 |
| Example 2 | 8.1 | 2 | 91 | 85 | −0.73 | 4.12 |
| Example 3 | 6.1 | 2 | 91 | 86 | 0.07 | 1.84 |
| Example 4 | 6.0 | 2 | 91 | 85 | 0.02 | 2.86 |
| Comparative | 5.9 | 2 | 88 | 82 | −0.76 | 5.01 |
| Example 4 | | | | | | |
| Comparative | 5.1 | 2 | 84 | 78 | 2.08 | 6.84 |
| Example 5 | | | | | | |

Table 1 shows the depolymerization efficiency and chromatic values of Comparative Example 1˜5 and Examples 1˜4. In this experiment, zinc chloride is used as the metal chloride, 1-Butyl-3-methylimidazolium chloride ([BMIM]Cl) is used as the alkylimidazole-chloride ionic liquid, and the bisalkylimidazole-metal tetrachloride ionic liquid is [Bmim]₂ZnCl₄. The chemical reaction occurs at a temperature of 80° C. for 5 hour. The chemical reaction is shown in the following Formula 2. After the reaction is completed, the bisalkylimidazole-zinc tetrachloride ionic liquid ([Bmim]₂ZnCl₄) as the graft chain is grafted on an activated carbon as the porous carrier to form the ion catalyst material.

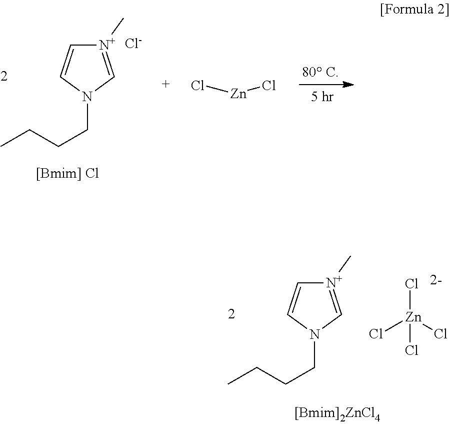

The PET chemical recycling is conducted as followings: adding the above PET chemical recycling ion catalyst materials to PET waste and ethylene glycol, followed by heating at a temperature of 196° C., and stirring with 120 rpm stirring speed for 4 hours to obtain depolymerized and decolorized bis(2-hydroxyethyl)terephthalate (BHET); and filtering and recycling the ion catalyst material for PET chemical recycling. A weight ratio of the PET waste to the ethylene glycol is 1:6. An addition amount of the ion catalyst material for PET chemical recycling is 0.5 wt %.

As shown in Table 1, Examples 1˜4 have the graft rates of 10.0 wt %, 8.1 wt %, 6.1 wt % and 6.0 wt %, falling within the claimed range of 6 wt % to 10 wt %. Comparative Examples 1˜3 have the graft rates of 11.4 wt %, 10.8 wt % and 10.1 wt %, above the claimed range of 6 wt % to 10 wt %. Comparative Examples 4 and 5 have the graft rates of 5.9 wt % and 5.1 wt %, below the claimed range of 6 wt % to 10 wt %. The experimental data show that the catalyst materials having the graft rate of 6 wt % to 10 wt % exhibit better depolymerization efficiency (>91%) than the Comparative Examples. In addition, Comparative Examples cannot meet the standard of chromatic values, comprising the L value from 85 to 90, the a value from −1 to 1, and the b value from 1 to 4. Especially, the b values of Comparative Example 3 (graft rate: 10.1 wt %) and Comparative Example 4 (graft rate: 5.9 wt %) are far from the desired scope: −4 to +4, while the b values of Example 1 (graft rate: 10.0 wt %) and Example 4 (graft rate: 6.0 wt %) fall within the desired range. These comparisons show that the claimed range of the graft rate plays a crucial role as an ion catalyst material in the capacity of depolymerization and decolorization. A slight change (±0.1%) around the boundary will cause a marked improvement for PET chemical recycling.

In summary, in the preparation method of an ion catalyst material for PET chemical recycling according to the disclosure, a porous material is used as a carrier so as to improve the decolorization ability with respect to PET waste (especially a textile fabric), and a bisalkylimidazole-metal tetrachloride ionic liquid is used so as to improve the depolymerization ability. In this way, in the disclosure, decolorization and depolymerization can be achieved and recycling can be facilitated by a simple manufacturing process. Accordingly, the problems of the related art, such as that depolymerization and decolorization need to be separately performed and recycling of a catalyst is not easy, can be effectively eliminated, and the cost can be reduced.

What is claimed is:

1. A preparation method of an ion catalyst material for PET chemical recycling, comprising:
adding a zinc chloride to a 1-Butyl-3-methylimidazolium chloride at a temperature of 60° C. to 120° C. for 1 hour to 6 hours to form a bisalkylimidazole-zinc tetrachloride ionic liquid that is grafted on a porous carrier,
wherein the porous carrier has a graft rate of 6 wt % to 10 wt % and the graft rate is the ratio of weight of the bisalkylimidazole-zinc tetrachloride ionic liquid to weight of the porous carrier,
wherein the porous carrier comprises activated carbon,
wherein a ratio of the zinc chloride to the 1-Butyl-3-methylimidazolium chloride is 1 to 2,
wherein a reaction of adding the zinc chloride to the 1-Butyl-3-methylimidazolium chloride to form the bisalkylimidazole-zinc tetrachloride ionic liquid is represented by following Formula 1,

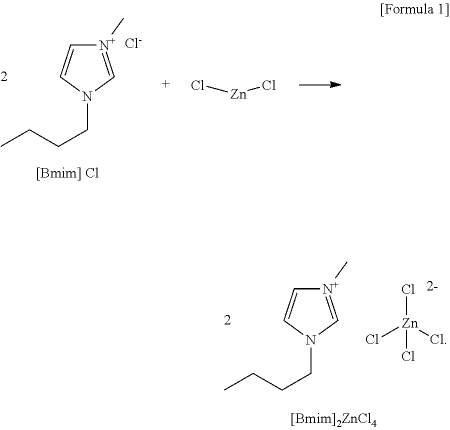

2. The preparation method of an ion catalyst material for PET chemical recycling according to claim 1, wherein the zinc chloride is added to the 1-Butyl-3-methylimidazolium chloride at 80° C. for 5 hours to form the bisalkylimidazole-zinc tetrachloride ionic liquid.

3. A PET chemical recycling method, comprising:
adding a PET chemical recycling ion catalyst material prepared by the preparation method of an ion catalyst material for PET chemical recycling according to claim 1 to PET waste and ethylene glycol, followed by heating and stirring to obtain depolymerized and decolorized bis(2-hydroxyethyl)terephthalate (BHET); and
filtering and recycling the ion catalyst material for PET chemical recycling.

4. The PET chemical recycling method according to claim 3, wherein the PET waste comprises a packaging material or a textile fabric.

5. The PET chemical recycling method according to claim 3, wherein a ratio of the PET waste to the ethylene glycol is 1:3 to 1:6.

6. The PET chemical recycling method according to claim 3, wherein an addition amount of the ion catalyst material for PET chemical recycling is 0.5% to 2%.

7. The PET chemical recycling method according to claim 3, wherein a heating temperature is 180° C. to 210° C., a stirring speed is 100 rpm to 150 rpm, and stirring time is 3 hours to 6 hours.
